(19) United States
(12) Patent Application Publication
(10) Pub. No.: US 2008/0207588 A1
(43) Pub. Date: Aug. 28, 2008

(54) Spiro-Heterocyclic Chromans, Thiochromans and Dihydroquinolines

(76) Inventors: Daniel T. W. Chu, Santa Clara, CA (US); Donald R James, El Sobrante, CA (US); Bing Wang, Cupertino, CA (US)

Correspondence Address: ELI LILLY & COMPANY, PATENT DIVISION, P.O. BOX 6288, INDIANAPOLIS, IN, 46206-6288, US

(21) Appl. No.: 11/814,878

(22) PCT Filed: Dec. 9, 2005
(86) PCT No.: PCT/US05/44713

§ 371 Date: Jul. 26, 2007

Related U.S. Application Data
(60) Provisional application No. 60/656,711, filed on Feb. 25, 2005.
(60) Provisional application No. 60/656,748, filed on Feb. 25, 2005.

(51) Int. Cl. A61K 31/337; C07D 305/14; C07D 205/12; A61K 31/438; A61P 11/00; A61P 19/02; A61P 35/00; A61K 31/397; C07D 215/00

(52) U.S. Cl. 514/210.19; 549/332; 548/950; 546/152; 514/449; 514/278

(57) ABSTRACT

The present invention is concerned with certain novel derivatives of Formula I,

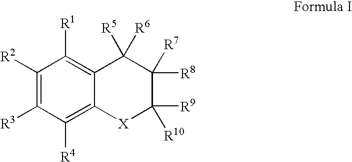

wherein R, R¹ to R¹⁰ are as described in the specification, and at least one CR⁵R⁶, or CR⁷R⁸, or CR⁹R¹⁰ is a an optionally substituted azetidine ring or an optionally substituted oxetan ring, which may be useful in the manufacture of pharmaceutical compositions for treating disorders mediated by lipoxygenases. They may also be useful in the manufacture of pharmaceutical formulations for the treatment of lipoxygenase mediated disorders.

### CROSS-REFERENCE TO RELATED APPLICATIONS

This application claims the benefit under 35 U.S.C. §119(e) of U.S. Provisional Application Ser. No. 60/656,748 filed on Feb. 25, 2005 and Ser. No. 60/656,711 also filed on Feb. 25, 2005, both of which are incorporated by reference in their entirety.

### BACKGROUND INFORMATION

The present invention relates to certain novel spiro-heterocyclic chroman, thiochroman and dihydroquinoline derivatives of Formula I as depicted below, pharmaceutical formulations containing them, and their uses as therapeutic agents, and syntheses therefore. Their uses as therapeutic agents that may act as lipoxygenase inhibitors include but are not limited to prevention or treatment of diseases involving apoptosis in cancer cells; diseases involving hypoxia or anoxia; diseases involving inflammation; disorders of the airways; and diseases involving the autoimmune system.

The use of compounds having a chroman moiety as lipoxygenase inhibitors has been disclosed, for example, in U.S. Pat. No. 5,059,609; U.S. Pat. No. 4,950,684; U.S. Pat. No. 5,015,661; U.S. Pat. No. 4,780,469; U.S. Pat. No. 5,591,772; U.S. Pat. No. 5,925,673; U.S. Pat. No. 5,250,547; U.S. Pat. No. 5,393,775; U.S. Pat. No. 4,814,346; U.S. Pat. No. 5,939,452, U.S. Pat. No. 6,051,601; U.S. Pat. No. 6,117,874; and U.S. Pat. No. 6,133,286.

Arachidonic acid is an essential fatty acid that exists within the cell membrane and can be released from phospholipids by the action of phospholipase. The released arachidonic acid is metabolized through three major enzymatic pathways, i.e. the lipoxygenase pathway, to form substances such as prostaglandins which are associated with inflammatory responses, and thromboxanes which are associated with the formation of thrombus, or leukotrienes which induce allergic reactions.

Lipoxygenases are non-heme iron-containing enzymes that catalyze the oxidation of polyunsaturated fatty acids and esters thereof. They were originally classified based on their substrate specificity for insertion of molecular oxygen into arachidonic acid at carbon positions 5, 12 and 15, but more recently a phylogenetic classification is being used. This separates the mammalian enzymes in four main subtypes, 5-Lipoxygenase, 12/15-Lipoxygenases, platelet 12-Lipoxygenases and epidermis-type lipoxygenases. The 12/15 family of lipoxygenases includes two sub-families with a high degree of sequence homology, the reticulocyte 15-Lipoxygenases (found in rabbit and humans) and the leukocyte 12-Lipoxygenases (found in mouse, pig, rat, and rabbit). This type of lipoxygenase shares more homology to reticulocyte 15-Lipoxygenase and leukocyte 12-Lipoxygenase, than to platelet 12-Lipoxygenases.

It is believed that oxidative metabolites of the 12/15-Lipoxygenase or the 15-Lipoxygenase cascade have been implicated in the potentiation of thrombin induced platelet activation (Setty et al. Blood, (1992), 2765-2773); in the progression of various cancers (Kelavkar et al, Curr. Urol. Rep. Vol. 3 no. 3 (2002),: pp. 207-214) and related pathologies (Tisdale et al., Science Vol. 289 no. 5488 (2000) pp. 2293-4). It has also been shown that treatment with a 15-Lipoxygenase inhibitor suppresses atherogenesis in rabbits fed a high-fat diet (Bocan et al., Atherosclerosis, Vol. 136 (1998) pp. 203-16). There is increasing evidence that certain lipoxygenase enzymes are involved in the pathogenesis and acceleration of atherosclerosis by inducing oxidation of LDL to its atherogenic form (Sparrow, C. P., et al., J. Lipid Res. Vol. 29 (1988) pp. 745-753. and Steinberg, D., New Eng. J. Med. Vol. 320(1989) pp. 915-924). It has also been reported that 12-Lipoxygenase enzyme plays a role in mediating angiotensin II induced vascular and adrenal actions (Natarajan, R., et al., Endocrinology Vol. 131 (1992) pp. 1174-1180). Recent studies (Klein, R. et al., Science Vol. 303 no. 5655 (2004) 329-332) have also shown the role of 15-Lipoxygenase enzyme in the regulation of bone density.

The enzyme 5-Lipoxygenase converts arachidonic acid to 5-hydroperoxyeicosatetraenoic acid (5-HPETE). This is the first step in the metabolic pathway yielding 5-hydroxyeicosatetraenoic acid (5-HETE) and the important class of mediators, the leukotrienes. Evidence of the role of leukotrienes in the pathology of certain diseases has been described, for example in Cloud et al., J. Allergy Clin. Immunol., Vol. 79 (1987) pp. 256 (asthma); Turnbull et al., Lancet II, (1977) pp. 526-9 (chronic bronchitis); Cromwell et al., Lancet II, (1981) pp. 164-5 (cystic fibrosis); Davidson et al., J. Pharm. Pharmacol. Vol. 34 no. 61(982) pp. 410 (rheumatoid arthritis); Rae et al., Lancet. Vol. 2 no. 8308 (1982) pp. 1122-4. Cook et al., J. Pharmacol. Exp. Ther., 235, (1985) pp. 470-474 (cardiovascular conditions); Tsuji et al., Biochem. Pharmacol. Vol. 55 no. 3: (1998); pp. 297-304 (dermatitis such as psoriasis).

It has also been shown in co-owned U.S. application Ser. No. 11/251,423 filed Oct. 13, 2005, titled Methods for Treating Diabetes, herein incorporated by reference in its entirety, that dual 5-Lipoxygenase and 12/15-Lipoxygenase inhibitors or 5-Lipoxygenase and 15-Lipoxygenase inhibitors are superior in the prevention of treatment of subjects susceptible to diabetes, are able to improve glucose control in animal models of diabetes, and have demonstrated a significant lowering of the baseline serum glucose levels compared to selective 5-Lipoxygenase, 15-Lipoxygenase and 12/15-Lipoxygenase inhibitors.

The compositions, formulations and methods of this invention are particularly applicable in preventing and/or treating diseases or disorders mediated, at least in part, by one or more lipoxygenase enzymes, such as 5-Lipoxygenase enzyme and/or 12/15-Lipoxygenase enzyme.

### SUMMARY OF THE INVENTION

The present invention is concerned with certain novel derivatives of Formula I, which may be useful in the manufacture of pharmaceutical compositions for treating disorders mediated by lipoxygenases.

In a first aspect, the present invention concerns the compounds represented by Formula

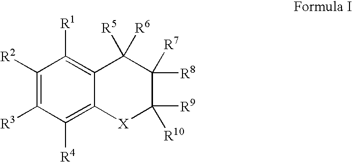

wherein,
X is O, S(O)₀₋₂, or NR;
R¹, R³, and R⁴ are independently selected from the group consisting of hydrogen, alkyl, cycloalkyl, halogen, nitro, cyano, amino, aminosulfonyl, sulfanyl, aryl, heterocyclyl, hydroxy, alkoxy, carboxy, alkoxycarbonyl, and aminocarbonyl;
R² is selected from the group consisting of hydroxy, amino, aminocarbonyl, alkoxy, —O-alkenyl, —O-acyl, —O-alkylene-amino, —O—C(O)-alkylene-COOR^{a}; —O—C(O)-alkylene-amino; —O—C(O)-alkylene-heterocyclyl; —O-glucoside; —O-phosphoryl, —O-alkylene-phosphoryl; —O—C(O)-AA, wherein AA is an amino acid, or a di-, tri- or tetra-peptide;
R⁵ and R⁶ are
   independently selected from the group consisting of hydrogen, alkyl, cycloalkyl, hydroxy, alkoxy, carboxy, alkylcarbonyl, amino, amido, aminosulfonyl, sulfonylamino, sulfanyl, nitro, cyano, halogen, —NR^{d}OR^{a}, and —NR^{d}—NR^{b}R^{c}; or
   together with the carbon atom to which they are attached form C═O, C═NOR^{a}, C═N—NR^{b}R^{c}, optionally substituted (C₃-C₈)cycloalkyl ring, an optionally substituted azetidine ring or an optionally substituted oxetan ring;
R⁷ and R⁸ are
   independently selected from the group consisting of hydrogen, alkyl, cycloalkyl, hydroxy, alkoxy, carboxy, alkylcarbonyl, amino, amido, aminosulfonyl, sulfonylamino, sulfanyl, nitro, cyano, halogen, —NR^{d}OR^{a}, and —NR^{d}—N^{b}R^{c}; or
   together with the carbon atom to which they are attached form C═O, C═NOR^{a}, C═N—NR^{b}R^{c}, optionally substituted (C₃-C₈)cycloalkyl ring, an optionally substituted azetidine ring or an optionally substituted oxetan ring;
R⁹ and R¹⁰ are
   independently selected from the group consisting of hydrogen, alkyl, cycloalkyl, hydroxy, alkoxy, carboxy, alkyl carbonyl, amino, amido, aminosulfonyl, sulfonylamino, sulfanyl, nitro, cyano, halogen, —NR^{d}OR^{a}, and —NR^{d}—NR^{b}R^{c}; or
   together with the carbon atom to which they are attached form C═O, C═NOR^{a}, C═N NR^{b}R^{c} an optionally substituted (C₃-C₈)cycloalkyl ring, an optionally substituted azetidine ring or an optionally substituted oxetan ring;
R is selected from the group consisting of hydrogen, alkyl, cycloalkyl, alkenyl, alkynyl, acyl, aminocarbonyl, heterocyclyl and aryl;
R^{a} is selected from the group consisting of hydrogen, alkyl, cycloalkyl, alkenyl, acyl, heterocyclyl, and aryl;
R^{b} and R^{c} are
   independently selected from the group consisting of hydrogen, alkyl, cycloalkyl, alkenyl, acyl, aminocarbonyl, heterocyclyl and aryl; or
   together with the nitrogen atom to which they are attached form an optionally substituted, saturated or unsaturated 3-8 membered ring optionally incorporating 1 to 3 N, O, or S atoms;
R^{d} is hydrogen or alkyl; and
with the proviso that
   at least one of —CR⁵R⁶, —CR⁷R⁸, and —CR⁹R¹⁰ is an optionally substituted azetidine ring or an optionally substituted oxetan ring;
or single stereoisomers, mixtures of stereoisomers, or pharmaceutically acceptable salts thereof.

In one embodiment, R² is hydroxy, and in another embodiment R² is hydroxy and R¹, R³, and R⁴ are independently of each other hydrogen, halogen, or alkyl. In yet another embodiment, R² is hydroxy, R¹, R³, and R⁴ are independently of each other hydrogen, halogen, or alkyl, and CR⁷R⁸ is an optionally substituted azetidine ring or an optionally substituted oxetan. In another embodiment, R² is hydroxy, R¹, R³, and R⁴ are independently of each other hydrogen, halogen, or alkyl, and CR⁹R¹⁰ is optionally substituted an optionally substituted azetidine ring; or an optionally substituted oxetan. In another embodiment, R² is hydroxy, R¹, R³, and R⁴ are independently of each other hydrogen, halogen, or alkyl, and CR⁵R⁶ is optionally substituted an optionally substituted azetidine ring; or an optionally substituted oxetan ring. In one embodiment R⁵ and R⁶ are both hydrogen.

In some embodiments, R⁵ is hydroxy, —NR^{d}OR^{a}, or —NR^{d}—NR^{b}R^{c}.

In one embodiment X is O or S. In another embodiment X is NR, R² is hydroxy, R³ is alkyl and R¹, R³ and R⁴ are independently of each other hydrogen or alkyl, and in a particular embodiment R is alkyl substituted with an amido, a sulfonylamino or an aminosulfonyl group and in another embodiment R is —(CH₂)₂₋₆—NR^{d}S(O)₂-aryl, —(CH₂)₂₋₆—S(O)₂NR^{d}-aryl; —(CH₂)₂₋₆NR^{d}C(O)-aryl or —(CH₂)₂₋₆—C(O)NR daryl; illustrated by alkylbenzenesulfonaminoethyl, or alkylbenzenesulfonaminopropyl.

It is contemplated that in embodiments where R² is selected from alkoxy, —O-alkenyl, —O-acyl, —O-alkylene-amino, —O—C(O)-alkylene-COOR^{a}; —O—C(O)-alkylene-amino; —O—C(O)-alkylene-heterocyclyl; —O-glucoside; —O-phosphoryl, —O-alkylene-phosphoryl; —O—C(O)-AA, wherein AA is an amino acid, or a di-, tri- or tetra-peptide, then the compound will hydrolyze in vivo to form the active hydroxy.

In another aspect, the invention relates to a pharmaceutical composition containing a therapeutically effective amount of a compound of Formula I, or a pharmaceutically acceptable salt thereof admixed with at least one pharmaceutically acceptable excipient. In some examples, the pharmaceutical compositions comprise a compound of Formula I and a pharmaceutically acceptable excipient; and the compound is selected from the illustrative compounds and stereoisomers, mixture of stereoisomers or pharmaceutically acceptable salts thereof.

In another aspect, the invention relates to a method of inhibiting a lipoxygenase, comprising contacting a cell with an effective amount of a compound of Formula I.

In one embodiment, the compound inhibits one or more lipoxygenase enzymes selected from 5-Lipoxygenase, 15-Lipoxygenase, 12/15-Lipoxygenase enzymes and combinations thereof. In some embodiments the compound inhibits 5-Lipoxygenase, and in other embodiments the compound inhibits both 5-Lipoxygenase and 15-Lipoxygenase or both 5-Lipoxygenase and 12/15-Lipoxygenase.

In some embodiments, the invention relates to a method of treating a subject with a lipoxygenase mediated disorder such as but not limited to apoptosis in cancer cells including prostatic cancer, gastric cancer, breast cancer, pancreatic cancer, colorectal or esophageal cancer and airways carcinoma; diseases involving hypoxia or anoxia including atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout, cardiopulmonary inflammation, rheumatoid arthritis, osteoarthritis, muscle fatigue and inflammatory disorders of the skin including acne, dermatitis and psoriasis; disorders of the airways including asthma, chronic bronchitis, human airway carcinomas, mucus hypersecretion, chronic obstructive pulmonary disease (COPD) pulmonary fibrosis caused by chemotherapy or other drugs, idiopathic pulmonary fibrosis, cystic fibrosis and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system including psoriasis, eczema, rheumatoid arthritis, and diabetes; and disorders involving bone loss or bone formation. In an illustrative example, the invention relates to a method of treating a subject with a lipoxygenase mediated disorder, such as but not limited to diabetes, arthritis, rheumatoid arthritis, chronic obstructive pulmonary disease (COPD), asthma, allergic rhinitis, Crohn's disease, and/or atherosclerosis.

In some of the embodiments, the compositions, methods of treatment and uses in the manufacture of pharmaceutical compositions therefor, may relate to compounds of Formula I such as:
5,7,8-trimethylspiro[chroman-2,3′-oxetan]-6-ol;
5,7-diethylspiro[chroman-2,3′-oxetan]-6-ol;
5,7-diethylspiro[chroman-2,2′-oxetan]-6-ol;
5′,7′-diethylspiro[azetidine-3,2′-chroman]-6′-ol;
5,7-diethyl-4-(methoxyamino)spiro[chroman-2,3′-oxetan]-6-ol;
5,7-diethyl-4-(methoxyamino)spiro[chroman-2,2′-oxetan]-6-ol;
5′,7′-diethyl-4′-(methoxyamino)spiro[azetidine-3,2′-chroman]-6′-ol;
5,7-diethylspiro[chroman-2,3′-oxetane]-4,6-diol;
5-ethyl-7-isopropylspiro[chroman-2, 3′-oxetane]-4,6-diol
7-isopropyl-5-methylspiro[chroman-2,3′-oxetane]-4,6-diol
5,7-diethyl-6-hydroxyspiro[chroman-2,3′-oxetan]-4-one;
5,7-diethylspiro[chroman-2,2′-oxetane]-4,6-diol;
5′,7′-dimethylspiro[azetidine-3,2′-chroman]-4′, 6′-diol;
5′,7′-diethyl-6′-hydroxyspiro[azetidine-3,2′-chroman]-4′-one;
7′-isopropyl-5′-methylspiro[azetidine-3,2′-chroman]-4′, 6′-diol
5,7-diethylspiro[chroman-4,3′-oxetan]-6-ol;
5′,7′-dimethylspiro[azetidine-3,4′-chroman]-6′-ol;
5,7-dimethylspiro[chroman-4,2′-oxetan]-6-ol;
5′,7′-dimethylspiro[azetidine-2,4′-chroman]-6′-ol;
5,7-dimethylspiro[chroman-3,3′-oxetan]-6-ol;
5′,7′-dimethylspiro[azetidine-3,3′-chroman]-6′-ol;
5′,7′-dimethyl-1′-methyl-2′,4′-dihydro-1′H-spiro[azetidine-3,3′-quinolin]-6′-ol;
5′,7′-dimethyl-1′-methyl-2′,4′-dihydro-1′H-spiro[oxetane-3,3′-quinolin]-6′-ol;
N-(3-(6′-hydroxy-5′,7′-dimethyl-2′,4′-dihydro-1′H-spiro[oxetane-3,3′-quinoline]-1′-yl)propyl)benzene-sulfonamide;
N-(3-(6′-hydroxy-5′,7′-dimethyl-2′,4′-dihydro-1′H-spiro[azetidine-3,3′-quinoline]-1′-yl)propyl)benzene-sulfonamide;
N-(3-(6′-hydroxy-5′,7′-dimethyl-2′,4′-dihydro-1′H-spiro[azetidine-3,3′-quinoline]-1′-yl)propyl)benzamide;
N-(3-(6′-hydroxy-5′,7′-dimethyl-2′,4′-dihydro-1′H-spiro[oxetane-3,3′-quinoline]-1′-yl)propyl)benzamide;
N-(3-(6′-hydroxy-5′,7′-dimethyl-2′,3′-dihydro-1′H-spiro[oxetane-3,4′-quinoline]-1′-yl)propyl)benzene-sulfonamide; and
N-(3-(6′-hydroxy-5′,7′-dimethyl-2′,3′-dihydro-1′H-spiro[azetidine-3,4′-quinoline]-1′-yl)propyl)benzene-sulfonamide.

Another aspect of this invention is the processes for preparing compounds of Formula I and is set forth in “Description of the Invention”.

### DETAILED DESCRIPTION OF THE INVENTION

### Definitions

As used in the present specification, the following words and phrases are generally intended to have the meanings as set forth below, except to the extent that the context in which they are used indicates otherwise.

The term “optional” or “optionally” means that the subsequently described event or circumstance may or may not occur, and that the description includes instances where said event or circumstance occurs and instances in which it does not.

It will be understood by those skilled in the art with respect to any group containing one or more substituents that such groups are not intended to introduce any substitution or substitution patterns that are sterically impractical and/or physically non-feasible.

The term “acyl” refers to the groups —C(O)—H, —C(O)-(alkyl), —C(O)-(cycloalkyl), —C(O)-(alkenyl), —C(O)-(cycloalkenyl), —C(O)-(aryl), and —C(O)-(heterocyclyl).

The term “acyloxy” refers to the moiety —O-acyl, including, for example, —O—C(O)-alkyl.

The term “alkenyl” refers to a monoradical branched or unbranched, unsaturated or polyunsaturated hydrocarbon chain, having from about 2 to 20 carbon atoms, for example 2 to 10 carbon atoms. This term is exemplified by groups such as ethenyl, but-2-enyl, 3-methyl-but-2-enyl (also referred to as “prenyl”, octa-2,6-dienyl, 3,7-dimethyl-octa-2,6-dienyl (also referred to as “geranyl”), and the like. The term also includes substituted alkenyl groups, and refers to an alkenyl group in which 1 or more, for example, 1 to 3 hydrogen atoms is replaced by a substituent independently selected from the group: ═O, ═S, acyl, acyloxy, alkoxy, amino (wherein the amino group may be a cyclic amine), aryl, heterocyclyl, carboxyl, carbonyl, amido, cyano, cycloalkyl, cycloalkenyl, halogen, hydroxyl, nitro, sulfamoyl (—SO₂NH₂), sulfanyl, sulfinyl (—S(O)H), sulfonyl (—SO₂H), and sulfonic acid (—SO₂OH). One of the optional substituents for alkenyl may be heterocyclyl, exemplified by 2-quinolyl-2-vinyl.

The term “alkenylene” refers to a diradical derived from the above defined monoradical, alkenyl.

The term “alkoxy” refers to the groups: —O-alkyl, —O-alkenyl, —O-cycloalkyl, —O-cycloalkenyl, and —O-alkynyl. Alkoxy groups that are —O-alkyl include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like. The term “alkoxy” also includes substituted alkoxy groups and refers to the groups —O-(substituted alkyl), —O-(substituted alkenyl), —O-(substituted cycloalkyl), —O-(substituted cycloalkenyl), —O-(substituted alkynyl) and —O-(optionally substituted alkylene)-alkoxy.

The term “alkyl” refers to a monoradical branched or unbranched saturated hydrocarbon chain having from about 1 to 20 carbon atoms. The term “alkyl” also means a combination of linear or branched and cyclic saturated hydrocarbon radical consisting solely of carbon and hydrogen atoms. This term is exemplified by groups such as methyl, ethyl, n-propyl, iso-propyl, n-butyl, iso-butyl, n-hexyl, n-decyl, tetradecyl, and the like. The term “alkyl” also includes substituted alkyl and refers to an alkyl group in which 1 or more, such as 1 to 5, hydrogen atoms is replaced by a substituent independently selected from the group: ═O, ═S, acyl, acyloxy, alkoxy, alkoxyamino, hydroxyamino, amino (wherein the amino group may be a cyclic amine), aryl, heterocyclyl, azido, carboxyl, alkoxycarbonyl, amido, cyano, cycloalkyl, cycloalkenyl, halogen, hydroxyl, nitro, sulfonylamino, aminosulfonyl, sulfanyl, sulfinyl, sulfonyl, and sulfonic acid. One of the optional substituents for alkyl may be hydroxy or amino, exemplified by hydroxyalkyl groups, such as 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and those compounds known as polyethylene glycols, polypropylene glycols and polybutylene glycols, and the like; or aminoalkyl groups exemplified by groups such as iaminomethyl, dimethylaminomethyl, diethylaminomethyl, ethylaminomethyl, piperidinylmethyl, morpholinylmethyl, and the like. Another substituent for alkyl may be halogen, such as trifluoromethyl. Another substituent may be hydroxyamino or alkoxyamino, exemplified by groups such as hydroxyaminomethyl, methoxyaminomethyl or ethoxyaminomethyl. Another substituent may be sulfanyl, exemplified by groups such as methyl (2-methylthioacetate). Another substituent may be aryl or heterocyclyl exemplified by methylbenzoate, propylisoindoline-1,3-dione, quinoline-methyl or 2-quinolyl-2-ethyl. Another substituent may be amido, aminosulfonyl or sulfonylamino, exemplified by 4-propylbenzensulfonamide-2-ethyl; 4-methylbenzene-sulfonamide-2-ethyl, 4-propylbenzensulfonamide-3-propyl; 4-methylbenzenesulfonamide-3-propyl, or methyl-N-methylacetamide. Another substituent may be aminocarbonyloxy (—OC(O)amino), such as —OC(O)NH₂ or —OC(O)-substituted amino.

The term “alkylene” refers to a diradical alkyl group, whereby alkyl is as defined above.

The term “alkynyl” refers to a monoradical branched or unbranched, unsaturated or polyunsaturated hydrocarbon chain, having from about 2 to 20 carbon atoms, for example 2 to 10 carbon atoms and comprising at least one triple bond, and preferably 1 to 3. The term also includes substituted alkynyl groups, and refers to an alkynyl group in which 1 or more hydrogen atoms is replaced by a substituent independently selected from the group: acyl, acyloxy, alkoxy, amino (wherein the amino group may be a cyclic amine), aryl, heterocyclyl, carboxyl, carbonyl, amido, cyano, cycloalkyl, cycloalkenyl, halogen, hydroxyl, nitro, sulfamoyl, sulfanyl, sulfinyl, sulfonyl, and sulfonic acid.

The term “amido” refers to the moieties —C(O)—NR¹⁰⁰R¹⁰¹ and —NR¹⁰⁰C(O)R¹⁰¹, wherein R¹⁰⁰ and R¹⁰¹ are independently selected from the group consisting of hydrogen, alkyl, substituted alkyl, alkenyl, alkynyl, cycloalkyl, cycloalkenyl, cycloalkynyl, and heterocyclyl, provided that R¹⁰⁰ and R¹⁰¹ are not aryl or heteroaryl.

The term “amino” refers to the group —NH₂ as well as to the substituted amines such as —NHR^{x} or —NR^{x}R^{x} where each R^{x} is independently selected from the group: alkyl, cycloalkyl, alkenyl, cycloalkenyl, alkynyl, aryl, heterocyclyl, acyl, optionally substituted alkoxy, carboxy and alkoxycarbonyl, and where —NR^{x}R^{x} may also be a cyclic saturated or unsaturated amine, optionally incorporating one or more, for example 1 to 3, additional atoms chosen form N, O or S, and optionally substituted with a substituent selected from the group consisting of ═O, ═S, alkyl, hydroxy, acyloxy, halo, cyano, nitro, sulfanyl, alkoxy, and phenyl. This term is exemplified by such groups as amino, cyclopropylamino, dimethylamino, diethylamino, hexylamino. The term “cyclic amine” or “cyclic amino” is exemplified by the group morpholinyl. The term “alkoxyamino” refers to embodiments wherein at least one of RX is alkoxy. The term “hydroxyamino” refers to embodiments wherein at least one of R^{x} is hydroxy.

“Amino acid” refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl or aralkyl (e.g., as in phenylalanine and tryptophan), substituted arylalkyl (e.g., as in tyrosine), and heteroarylalkyl (e.g., as in histidine). The term “naturally occurring amino acids” refers to these amino acids.

Unnatural amino acids are also known in the art, as set forth in, for example, Williams (ed.), Synthesis of Optically Active .alpha.-Amino Acids, Pergamon Press (1989); Evans et al., J. Amer. Chem. Soc., 112:4011-4030 (1990); Pu et al., J. Org Chem., 56:1280-1283 (1991); Williams et al., J. Amer. Chem. Soc., 113:9276-9286 (1991); and all references cited therein.

The term “peptide” refers to any of various natural or synthetic compounds containing two or more amino acids linked by the carboxyl group of one amino acid to the amino group of another. A “dipeptide” refers to a peptide that contains 2 amino acids. A “tripeptide” refers to a peptide that contains 3 amino acids. A “tetrapeptide” refers to a peptide that contains 4 amino acids.

The term “aromatic” refers to a cyclic or polycyclic moiety having a conjugated unsaturated (4n+2) π electron system (where n is a positive integer), sometimes referred to as a delocalized n electron system.

The term “aryl” refers to an aromatic cyclic hydrocarbon group of from 6 to 20 carbon atoms having a single ring (e.g., phenyl) or multiple condensed (fused) rings (e.g., naphthyl or anthryl). Aryls include phenyl, naphthyl and the like. The term “aryl” also includes substituted aryl rings and refers to an aryl group as defined above, which unless otherwise constrained by the definition for the aryl substituent, is substituted with one or more, such as 1 to 5, substituents, independently selected from the group consisting of: hydroxy, acyl, acyloxy, alkenyl, alkoxy, alkyl, alkynyl, amino, aryl, aryloxy, azido, carboxyl, alkoxycarbonyl, amido, cyano, cycloalkyl, cycloalkenyl, halogen, heterocyclyl, heterocyclyloxy, nitro, sulfonylamino, aminosulfonyl, sulfanyl, sulfinyl, sulfonyl, and sulfonic acid.

The term “aryloxy” refers to the group —O-aryl.

The term “aralkyl” refers to the group -alkylene-aryl, wherein alkylene and aryl are defined herein.

The term “carbonyl” refers to the di-radical “C═O”, which is also illustrated as “—C(O)—”. This moiety is also referred as “keto.”

The term “alkylcarbonyl” refers to the groups: —C(O)-(alkyl), —C(O)-(cycloalkyl), —C(O)-(alkenyl), and —C(O)-(alkynyl).

The term “alkoxycarbonyl” refers to the groups: —C(O)O-(alkyl), —C(O)O-(cycloalkyl), —C(O)O-(alkenyl), and —C(O)O-(alkynyl). These moieties may also be referred to as esters.

The term “aminosulfonyl” refers to the group —S(O)₂-(amino). The term “sulfonylamino” refers to the group -(amino) —S(O)₂-R^{y}, wherein R^{y} is alkyl, cycloalkyl, alkenyl, aryl or heterocyclyl.

The term “aminocarbonyl” refers to the group —C(O)-( amino) and the term “cabonylamino” refers to the group -amino-C(O)-R^{y}, wherein R^{y} is alkyl, cycloalkyl, alkenyl, aryl or heterocyclyl and the term amino is as described herein.

The term “carboxy” or “carboxyl” refers to the moiety “—C(O)OH,” which is also illustrated as “—COOH.” The salts of —COOH are also included.

The term “cycloalkyl” refers to non-aromatic cyclic hydrocarbon groups having about 3 to 12 carbon atoms having a single ring or multiple condensed or bridged rings. Such cycloalkyl groups include, by way of example, single ring structures such as cyclopropyl, cyclobutyl, cyclopentyl, cyclohexyl, and the like, or multiple ring structures such as adamantyl, and the like. The term “cycloalkyl” additionally encompasses spiro systems wherein the cycloalkyl ring has a carbon ring atom in common with another ring. The term “cycloalkyl” also includes substituted cycloalkyl rings and refers to a cycloalkyl group substituted with one or more, such as 1 to 5, substituents, independently selected from the group consisting of: ═O, ═S, acyl, acyloxy, alkenyl, alkoxy, alkyl, alkynyl, amino, aryl, aryloxy, azido, carboxyl, alkoxycarbonyl, amido, cyano, cycloalkyl, cycloalkenyl, halogen, heterocyclyl, heterocyclyloxy, hydroxyl, nitro, sulfonylamino, aminosulfonyl, sulfanyl, sulfinyl, sulfonyl, and sulfonic acid. A cycloalkyl ring substituted with an alkyl group is also referred as “alkylcycloalkyl.”

The term “cycloalkenyl” refers to cyclic alkenyl groups of from 3 to 10 carbon atoms having single or multiple cyclic rings. This also includes substituted cycloalkenyl which includes substituents as those listed with cycloalkyl.

The term “halo” or “halogen” refers to fluoro, chloro, bromo, and iodo.

The term “heteroaryl” refers to an aromatic carbocyclic radical having one or more, such as 1 to 3, rings incorporating one or more, such as 1 to 4, heteroatoms within the ring (chosen from nitrogen, oxygen, and/or sulfur). This term excludes saturated carbocyclic radical having one or more rings incorporating one or more heteroatoms within the ring (chosen from nitrogen, oxygen, and/or sulfur).

The terms “heterocycle,” “heterocyclic,” “heterocyclo,” and “heterocyclyl” refer to a monovalent, saturated, partially unsaturated or fully unsaturated (aromatic) carbocyclic radical having one or more, such as 1 to 3, rings incorporating one or more, such as 1 to 4, heteroatoms within the ring (chosen from nitrogen, oxygen, and/or sulfur). Heterocycles include morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, quinuclidine, and the like. The terms “heterocycle,” “heterocyclic,” “heterocyclo,” and “heterocyclyl” also include substituted rings and refer to a heterocycle group as defined above, which unless otherwise constrained by the definition for the heterocycle, is substituted with one or more, such as 1 to 5, substituents, independently selected from the group consisting of: hydroxy, acyl, acyloxy, alkenyl, alkoxy, alkyl, alkynyl, amino, aryl, aryloxy, azido, carboxyl, alkoxycarbonyl, amido, cyano, cycloalkyl, cycloalkenyl, halogen, heterocyclyl, heterocyclo-oxy, nitro, sulfonylamino, aminosulfonyl, sulfanyl, sulfinyl, sulfonyl, and sulfonic acid. This term is exemplified by 4,5-dihydroisoxazole-5-methylcarboxylate, 5-butylisoxazol, pyrrolidinyl, morpholinyl, imidazolyl, 5-hydroxypyridin-2-yl, dimethylaminopyridin-3-yl, isoindol inedione, trifluoromethyloxazolyl, 2-bromophenyl-1H-tetrazol-5-yl, methylthiazolyl, phenylthiazolyl, and benzothiazolyl.

The term “heterocyclyloxy” refers to the moiety —O-heterocyclyl.

The term “inflammation,” “inflammatory conditions,” or “inflammation conditions” includes but is not limited to muscle fatigue, osteoarthritis, rheumatoid arthritis, inflammatory bowel syndrome or disorder, Crohn's disease, skin inflammation, such as atopic dermatitis, contact dermatitis, allergic dermatitis, xerosis, eczema, rosacea, seborrhea, psoriasis, atherosclerosis, thermal and radiation burns, acne, oily skin, wrinkles, excessive cellulite, excessive pore size, intrinsic skin aging, photo aging, photo damage, harmful UV damage, keratinization abnormalities, irritation including retinoid induced irritation, hirsutism, alopecia, dyspigmentation, inflammation due to wounds, scarring or stretch marks, loss of elasticity, skin atrophy, and gingivitis.

The term “ischemia” refers to deficiency of blood to an organ or tissue due to functional constriction or actual obstruction of a blood vessel.

The term “isomers” or “stereoisomers” relates to compounds that have identical molecular formulae but that differ in the arrangement of their atoms in space. Stereoisomers that are not mirror images of one another are termed “diastereoisomers” and stereoisomers that are non-superimposable mirror images are termed “enantiomers,” or sometimes optical isomers. A mixture of equal amounts of stereoisomers of a molecule is termed a “racemate” or a “racemic mixture.” A carbon atom bonded to four non-identical substituents is termed a “chiral center.” Certain compounds of the present invention have one or more chiral centers and therefore may exist as either individual stereoisomers or as a mixture of stereoisomers. Configurations of stereoisomers that owe their existence to hindered rotation about double bonds are differentiated by their prefixes cis and trans, (or Z and E), which indicate that the groups are on the same side (cis or Z) or on opposite sides (trans or E) of the double bond in the molecule according to the Cahn-Ingold-Prelog rules. This invention includes all possible stereoisomers as individual stereoisomers, racemates, or mixtures of stereoisomers.

A “lipoxygenase-mediated condition” or a “disorder mediated by lipoxygenases” means any condition, disorder or disease mediated, at least in part, by a lipoxygenase enzyme. This includes disorders related to or otherwise associated with a lipoxygenase enzyme or the inhibition thereof, including, by way of example and without limitation, diseases involving apoptosis in cancer cells such as prostatic cancer, gastric cancer, breast cancer, pancreatic cancer, colorectal or esophageal cancer and airways carcinoma; diseases involving hypoxia, or anoxia such as atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout; cardiopulmonary inflammation, rheumatoid arthritis, osteoarthritis, muscle fatigue and inflammatory disorders of the skin including acne, dermatitis and psoriasis; disorders of the airways such as asthma, chronic bronchitis, human airway carcinomas, mucus hypersecretion, chronic obstructive pulmonary disease (COPD), pulmonary fibrosis caused by chemotherapy or other drugs, idiopathic pulmonary fibrosis, cystic fibrosis, and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system such as psoriasis, eczema, rheumatoid arthritis, and diabetes; and disorders involving bone loss or bone formation.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts which retain the biological effectiveness and properties of the compounds of this invention and which are not biologically or otherwise undesirable. In some cases, the compounds of this invention are capable of forming acid and/or base salts by virtue of the presence of phenolic, amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heterocyclic, and the like. Also included are amines where the two or three substituents, together with the amino nitrogen, form a heterocyclic group.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

It should be understood that for the purpose of this invention, all references to acceptable salts also include solvent addition forms (solvates) or polymorphs (crystal forms). “Solvate” means solvent addition form that contains either stoichiometric or non-stoichiometric amounts of solvent. Some compounds have a tendency to trap a fixed molar ratio of solvent molecules in the crystalline solid state, thus forming a solvate. If the solvent is water the solvate formed is a “hydrate,” when the solvent is alcohol, the solvate formed is an “alcoholate.” “Polymorphs” (or “crystal forms”) means crystal structures in which a compound can crystallize in different crystal packing arrangements, all of which have the same elemental composition. Different crystal forms usually have different X-ray diffraction patterns, infrared spectra, melting points, density, hardness, crystal shape, optical and electrical properties, stability and solubility. Recrystallization solvent, rate of crystallization, storage temperature, and other factors may cause one crystal form to dominate.

The term “prodrug” refers to an inactive form of a compound which must be metabolized in vivo, e.g., by biological fluids or enzymes, by a subject after administration into an active form of the parent compound in order to produce the desired pharmacological effect. The prodrug can be metabolized before absorption, during absorption, after absorption, or at a specific site. Prodrug forms of compounds may be utilized, for example, to improve bioavailability, improve subject acceptability such as masking or reducing unpleasant characteristics such as a bitter taste, odor, or gastrointestinal irritability, alter solubility, provide for prolonged or sustained release or delivery, improve ease of formulation, or provide site-specific delivery of the compound.

Prodrugs of a compound of this invention are prepared by modifying one or more functional group(s) present in the compound in such a way that the modification(s) may be cleaved in vivo to release the parent compound. Prodrugs include compounds wherein a hydroxyl group in a compound of the invention is bonded to any group that may be cleaved in vivo to regenerate the free hydroxyl, amino. Examples of prodrugs include, but are not limited to, esters (e.g., acetate, formate, and benzoate derivatives), carbamates (e.g., N,N-dimethylaminocarbonyl) of hydroxy functional groups in compounds of the invention, see Bundegaard, H. Design of Prodrugs. New York-Oxford: Elsevier, 1985, pp. 1-92., and the like. Reference to a compound herein includes prodrug forms of said compound.

The term “subject” includes, but is not limited to, humans and animals, such as farm animals (cattle, horses, sheep, goats, and swine) and domestic animals (rabbits, dogs, cats, rats, mice and guinea pigs. The term “subject” does not denote a particular age or sex.

The term “sulfanyl” or “thio” refers to the groups: —S—H, —S-(alkyl), —S-(aryl), or —S-(heterocyclyl). The term is exemplified by groups such as isopropylthio and methyl thioacetate.

The term “therapeutically effective amount” refers to that amount of a compound of this invention that is sufficient to effect treatment, as defined below, when administered to a subject in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can readily be determined by one of ordinary skill in the art.

The term “treatment” or “treating” means any treatment of a disease or disorder in a subject, including:
preventing or protecting against the disease or disorder, that is, causing the clinical symptoms not to develop;
inhibiting the disease or disorder, that is, arresting or suppressing the development of clinical symptoms; and/or
relieving the disease or disorder that is, causing the regression of clinical symptoms.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

### Nomenclature

In general, the nomenclature used in this Application was generated using or with the help of the naming package within the ChemDrawUltra® version 9.0.1 suite of programs by CambridgeSoft Corp. (Cambridge, Mass.).

### Synthesis of the Compounds of the Invention

### Synthetic Reaction Parameters

The terms “solvent”, “inert organic solvent” or “inert solvent” mean a solvent inert under the conditions of the reaction being described in conjunction therewith. Solvents employed in synthesis of the compounds of the invention include, for example, methanol (“MeOH”), acetone, water, acetonitrile, 1,4-dioxane, dimethylformamide (“DMF”), benzene, toluene, tetrahydrofuran (“THF”), chloroform, methylene chloride (also named dichloromethane (“DCM”)), diethyl ether, ethyl acetate (“EtOAc”), pyridine and the like, as well as mixtures thereof. Unless specified to the contrary, the solvents used in the reactions of the present invention are inert organic solvents.

Unless specified to the contrary, the reactions described herein take place at atmospheric pressure within a temperature range from −10° C. to 110° C. and in some cases at “room” or “ambient” temperature, e.g., 20° C. Further, unless otherwise specified, the reaction times and conditions are intended to be approximate.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

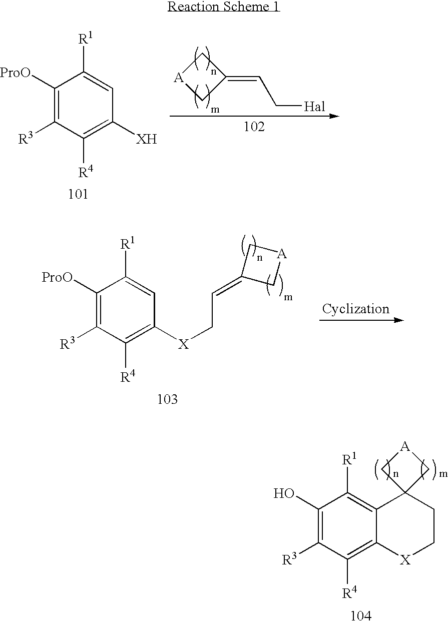

Scheme 1 describes a synthesis for compounds of Formula I of the present invention wherein X═S, CR⁵R⁶ form an azetidine or an oxetan ring, R⁷, R⁸, R⁹, R¹⁰ are hydrogen, and R¹, R³, R⁴ are as described herein. Compound 101 can be treated with an allyl halide of formula 102, wherein Hal is halide such as a bromide, A is a heteroatom such as O or N, n is 0 to 2, m is 0 to 2, and n+m=2, to give a compound 103 wherein Pro is a protective group. Intramolecular cyclization catalyzed with a Lewis acid such as boron trifluoride, followed by deprotection may yield a compound 104 wherein A is a heteroatom such as O or N, n is 0 to 2, m is 0 or 2, and n+m=2.

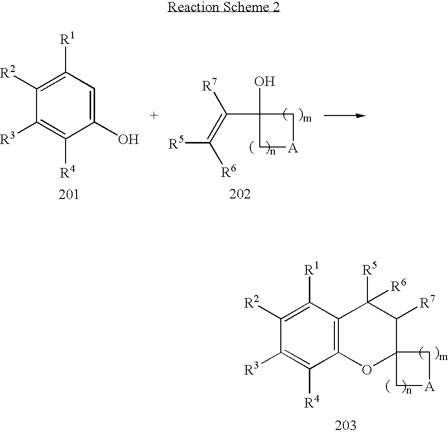

Scheme 2 describes a synthesis for compounds of Formula I, wherein X is O, R⁹ and R¹⁰ form an azetidine or an oxetan ring, R⁸ is hydrogen, and R¹ to R⁴, and R⁵ to R⁷ are as described herein. The vinyl-cycloalcohol of formula 202, which may be prepared by Grignard reaction of a vinyl magnesium bromide of formula CR⁵R⁶═CR⁷MgBr with azetidinone or an oxetanone, under inert conditions in a solvent such as tetrahydrofuran, may react with the phenol of formula 201 in the presence of a Lewis acid, such as BF₃-ether, methane sulfonic acid, p-toluene sulfonic acid, or aluminum chloride, to give the compound of formula 203, wherein A is a heteroatom such as O or N, n is 0 to 2, m is 0 to 2, and n+m=2.

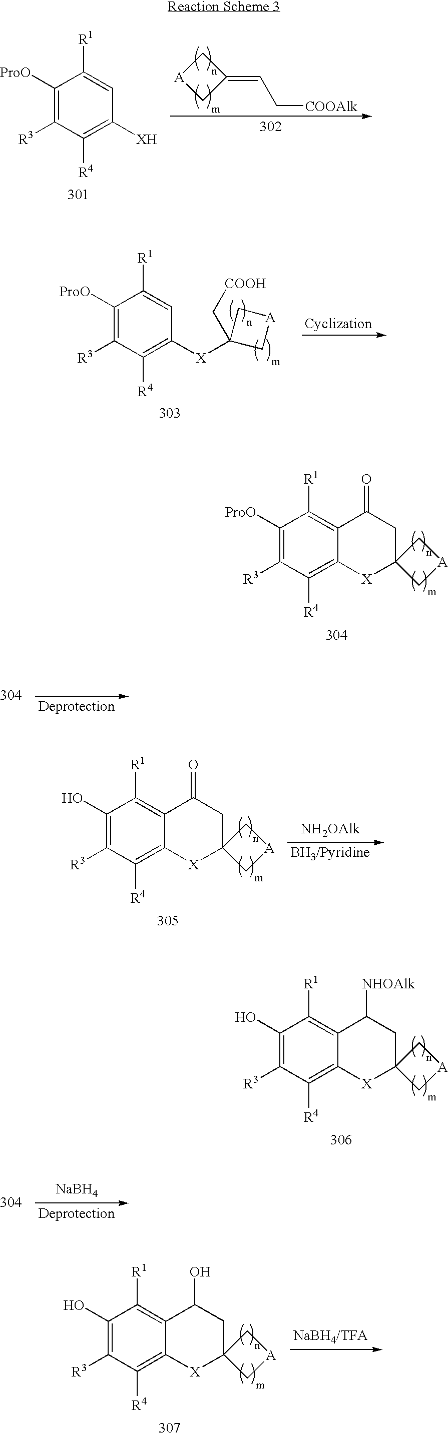

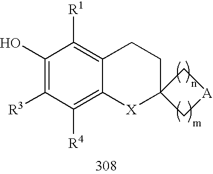

Scheme 3 describes the synthesis for compounds of Formula I wherein R⁹ and R¹⁰ form an azetidine or an oxetan ring; R⁵, R⁶, R⁷, R⁸ are hydrogen, R² is hydroxy, R¹, R³, and R⁴ are as described herein, and X is S or NR, starting from compound of formula 301, wherein Pro is a protective group, A is a heteroatom such as O or N, n is 0 to 2, m is 0 to 2, and n+m=2, and treating it with a cycloalkylidene carboxylate of formula 302 to give a compound of formula 303. Intramolecular cyclization in the presence of a Lewis acid may give compound of formula 304. Deprotection of compound 304 may give the compound 305 that can be treated with alkoxyamine followed by reduction with borane/pyridine complex to give a compound of formula 306. Compound 304 may also be reduced with sodium borohydride followed by deprotection to yield compound of formula 307, which can be further reduced under acidic conditions, such as with sodium borohydride in the presence of trifluoroacetic aced to yield a compound of formula 308.

If X is NH, any of the compounds of formula 305, 306, 307 or 308 can be coupled with a compound RL wherein R is as described herein but not hydrogen, and L is a leaving group such as an halide for example a chloride, a bromide or an iodide to yield a derivative of Formula I wherein X is substituted nitrogen.

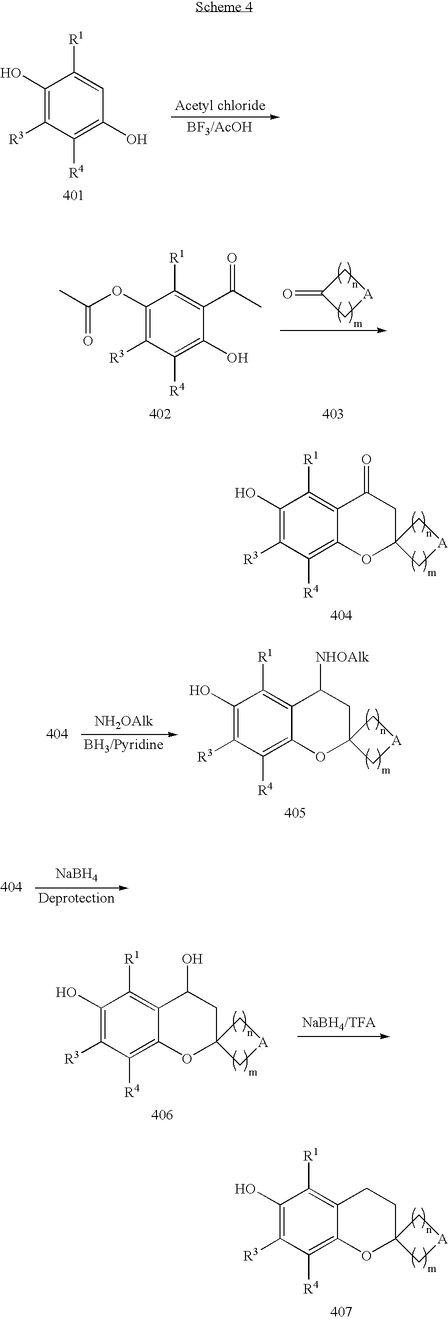

Scheme 4 describes the synthesis for compounds of Formula I, wherein R⁹ and R¹⁰ form an azetidine or an oxetan ring, R⁵, R⁶, R⁷, R⁸, are hydrogen and X is O, starting from a hydroquinone of formula 401 wherein R¹, R³ and R⁴ are as described herein, and treating it with acetylchloride to give the diacetate which after work-up was further treated with boron trifluoride-acetic acid complex in the presence of pyrrolidine to give the acetylacetate of formula 402. Treatment with azetidinone or oxetanone of formula 403 wherein A is a heteroatom such as O or N, n is 0 to 2, m is 0 to 2, and n+m=2, may give a compound of formula 404 that can be treated with alkoxyamine followed by reduction with sodium/pyridine complex to give a compound 405. compound 404 may also be reduced with sodium borohydride in methanol to yield a compound 406, which can be further reduced under acidic conditions such as with sodium borohydride in the presence of trifluoroacetic aced to yield a compound of formula 407.

### Preferred Compounds

The compounds of Formula I encompass the derivatives of the invention as disclosed, and/or the pharmaceutically acceptable salts of such compounds. In addition, the compounds of this invention include the individual stereochemical isomers and mixtures thereof, arising from the selection of substituent groups. It will be understood by those skilled in the art with respect to any group containing one or more substituents that such groups are not intended to introduce any substitution or substitution patterns that are sterically impractical and/or synthetically non-feasible.

### Utility, Testing and Administration

### General Utility

Without subscribing to a particular theory or mechanism of action, compounds of the invention may target certain enzymes known as “oxidoreductases” that function widely across a variety of physiological processes, for example, certain compounds of the present invention may target lipoxygenases such as 5-Lipoxygenase, 12-Lipoxygenase, 15-Lipoxygenase, and/or 12/15-Lipoxygenase. In particular, oxidoreductases catalyze reactions in which two molecules interact so that one molecule is oxidized and the other is reduced. Alterations in oxidoreductases are thought to account for as many as 3% of all known human genetic diseases. Abnormalities in oxidoreductase activity may underlie such disorders as congestive heart failure, respiratory chain defects (e.g., abnormalities associated with enzymes of the respiratory chain, acute respiratory distress syndrome (ARDS)), glycogen storage disease, end-stage renal disease, and rheumatoid arthritis. Inhibitors of lipoxygenases are known to be useful in the prevention or treatment of, for example, disorders selected from apoptosis in cancer cells including prostatic cancer, gastric cancer, breast cancer, pancreatic cancer, colorectal or esophageal cancer and airways carcinoma; diseases involving hypoxia or anoxia, including atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout, cardiopulmonary inflammation, rheumatoid arthritis, osteoarthritis, muscle fatigue and inflammatory disorders of the skin including acne, dermatitis and psoriasis; disorders of the airways including asthma, chronic bronchitis, human airway carcinomas, mucus hypersecretion, chronic obstructive pulmonary disease (COPD), pulmonary fibrosis caused by chemotherapy or other drugs, idiopathic pulmonary fibrosis, cystic fibrosis, and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system including psoriasis, eczema, rheumatoid arthritis, and diabetes; and disorders involving bone loss or bone formation

Certain compounds of the present invention are also useful in treating conditions falling with the group of dermatologic conditions, such as prevention and protection of skin tissue against age-related damage or damage resulting from insults such as harmful ultraviolet (UV) radiation, use of retinoids, wearing diapers, stress and fatigue, and in the treatment of contact dermatitis, skin irritation, skin pigmentation, psoriasis, or acne. Testing

This section describes how compositions incorporating compositions of the present invention are selected, using in vitro and/or in vivo models, and used as therapeutic interventions in the exemplary indications in support of the present invention.

The 5-Lipoxygenase pathway is a major synthetic pathway relevant to human inflammatory disease. The enzyme 5-Lipoxygenase catalyses the two first steps in the oxygenation of arachidonic acid (a polyunsaturated 20-carbon fatty acid) to leukotrienes. Leukotrienes are known to be important mediators of inflammatory and allergic reactions. The first step in the synthesis of leukotrienes, which is catalyzed by 5-Lipoxygenase, is the formation of 5-HPETE. The rearrangement of 5-HPETE to form the unstable LTA₄, the rate-limiting step in the synthesis of the leukotrienes, is also catalyzed by 5-Lipoxygenase. LTA₄ is then converted to either LTB₄ or LTC₄. LTC₄ is rapidly metabolized to LTD₄ and then to LTE₄. LTC₄, LTD₄ and LTE₄ are collectively referred to as the cysteinyl (Cys) leukotrienes.

Biosynthesis of LTB₄, LTC₄, LTD₄ and LTE₄ occurs predominantly in leukocytes, in response to a variety of immunological stimuli. The primary target of LTB₄ is the leukocyte where it elicits enzyme release, chemotaxis, adherence, and aggregation in nM concentrations. LTB₄ modulates immune responses and participates in the host-defense against infections. Hence, LTB₄ is an important chemical mediator in the development and maintenance of inflammatory reactions and disease states.

Endogenous lipoxygenase metabolites may also be involved in enhanced cytokine tumor necrosis factor α (TNF-α) production following certain stimuli such as silica, asbestos and lipopolysaccharides (Rola-Pleszczynski, M et al. Mediators of Inflammation 1 : 5-8 (1992)). Consistent with selective lipoxygenase inhibitory effect, certain compounds of the present invention have also shown to have an inhibitory effect on TNF-α. synthesis and/or release. The “TNF-α” has a broad spectrum of biological activities, plays an important role in coordinating the body's response to infection, and serves as an important mediator of inflammation. It is known that inflammatory cytokines have been shown to be pathogenic in several diseases including, but not limited to asthma (N. M. Cembrzynska et al., Am. Rev. Respir. Dis., 147, 291 (1993)), Adult Respiratory Distress Syndrome (ARDS). (Miller et al., Lancet 2 (8665); 712-714 (1989) and Ferrai-Baliviera et al., Arch. Surg. 124 (12): 1400-1405 (1989)), lung fibrosis (Piguet et al., Nature, 344:245-247 (1990) and Bissonnette et al., Inflammation 13 (3): 329-339 (1989)), bone resorption diseases (Bertolini et al., Nature 319: 516-518 (1986) and Johnson et al., Endocrinology 124 (3): 1424-1427 (1989)), auto-immune diseases (W. Fiers, FEBS Lett., 1991, 285, p. 199). It will be therefore appreciated that compounds of the present invention showing an inhibitory effect on both 5-Lipoxygenase and TNF-α should be superior in the treatment or amelioration of for example diseases such as respiratory disorders, antiprolilferative disorders or autoimmune disorders.

In vitro evaluation of the ability of a composition to inhibit the enzymes 5-Lipoxygenase, 15-Lipoxygenase, or 12/15-Lipoxygenase as described in Walidge, N. B. et al. Anal. Biochem., Vol. 231 (1995), pp. 354-358 using a high throughput colorimetric method; as well as in vitro evaluation of inhibiting LTB₄ is described in Examples.

In vitro cell-based assays for inflammation are well known in the art, for example, e-selectin (also named Endothelial Leukocyte Adhesion Molecule or ELAM) or C-reactive protein (CRP). The ELAM assay measures in vitro activity of the test compounds in reducing expression of ELAM in activated endothelial cells. Briefly, endothelial cells are created by adding known activators such as lipopolysaccharides, TNF or IL-1β, alone or in some combination. Activated cells produce ELAM, which can be measured using, for example, an E-selectin monoclonal antibody-based ELISA assay.

In vivo evaluation of anti-inflammatory activity can be determined by well characterized assays measuring Carrageenan-Induced Paw Edema, by Mouse Ear Inflammatory Response to Topical Arachidonic Acid (Gabor, M. Mouse Ear Inflammation Models and their Pharmacological Applications (2000)), or by the in vivo murine Zymosan peritonitis assay. Carrageenan-Induced Paw Edema is a model of inflammation, which causes time-dependent edema formation following carrageenan administration into the intraplantar surface of a rat paw. The application of arachidonic acid to the ears of mice produces immediate vasodilation and erythema, followed by the abrupt development of edema, which is maximal at 40 to 60 min. The onset of edema coincides with the extravasations of protein and leukocytes. After one hour the edema wanes rapidly and the inflammatory cells leave the tissue so that at 6 hours the ears have returned to near normal.

Administration of Zymosan-A, a purified polysaccharide fraction of yeast cell wall has been used since the 1980s to induce acute inflammatory response in rodents. The inflammatory response is characterized by marked induction of pro-inflammatory cytokines, influx of inflammatory cells and biosynthesis of arachidonic acid metabolites as early as five minutes after the Zymosan injection. The purpose of this model is to evaluate the ability of compounds to reduce inflammatory response induced by administration of Zymosan-A and assessed by the level of inflammatory cytokines and arachidonic metabolites in the fluid exudates.

These assays, as described in the Examples, measure a test compound's ability to treat these inflammatory processes via systemic and topical routes of administration.

Protection against redox stress can be evaluated in cell culture using high glutamate induced oxidative stress (HGOS) in mouse dopaminergic cell lines. The cytotoxic effect of glutamate is not due to excitotoxicity, as this cell line is devoid of inotropic glutamate receptors. Rather, the glutamate-induced toxicity of dopaminergic cells is associated with an inhibition of cystine transport which subsequently leads to depletion of intracellular glutathione (GSH) levels (Murphy T. H., et al. Neuron, Vol. 2 (1989), pp. 1547-1558), activation of neuronal 12-Lipoxygenase (Li, Y. et al. Neuron, Vol. 19 (1997), pp. 453-463), increased ROS production (Tan S. et al. J. Cell Biol., Vol.141 (1998), pp. 1423-1432) and elevated intracellular Ca²⁺ (Li, Y. et al. see supra). Some molecules were measured for their ability to protect cells against glutamate-induced stress and the assay is detailed in Examples.

Further validation of neuroantiinflammatory activity of compounds can be assessed in vitro by the inhibition of IL-1.beta. release from a microglial cell line.

Interleukin-1 (IL-1) is a pro-inflammatory cytokine that exists in two separate forms that share 30% sequence homology (alpha and beta). Constitutive expression of IL-1 is low in the brain but levels of both forms of this cytokine increase dramatically after injury. There is substantial evidence that IL-1 is an important mediator of neurodegeneration induced by cerebral ischemia (Touzani, O. et al. J. Neuroimmunol., Vol.100 (1999), pp. 203-215). Both IL-1 forms are rapidly induced in experimental models of stroke and administration of recombinant IL-1β enhances ischemic injury (see Hill J. K., et al. Brain Res., Vol. 820 (1999), pp. 45-54); Hillhouse E. W. et al. Neurosci. Lett. Vol. 249 (1998), pp. 177-179; Loddick S. A. et. al. J. Cereb. Blood Flow Metab. Vol. 16 (1996), pp. :932-940; Stroemer R. P. et al. J. Cereb. Blood Flow Metab. Vol. 18 (1998), pp. 833-839). Conversely, blocking IL-1 actions with a receptor antagonist or a neutralizing antibody markedly reduces neuronal death and inflammation in models of ischemic damage (see Betz, A. L., J. Cereb. Blood Flow Metab. Vol. 15 (1995), pp. 547-551; Relton, J. K., Brain Res. Bull. Vol. 29 (1992), pp. 243-246; Yamasaki, Y. et al. Stroke, Vol. 26 (1995), pp. 676-680). Furthermore, mice with decreased IL-1β production (caspase-1 knockouts) are significantly protected from ischemic injury (Schielke, G. P. et al. J. Cereb. Blood Flow Metab. Vol. 18 (1998), pp. 180-185) and IL-1^{α} and β double knockouts exhibit dramatically reduced ischemic infarct volumes compared with wild-type mice (87% reduction in cortex) (Boutin, H. et al. J. Neurosci. Vol. 21 (2001), pp. 5528-5534).

In addition to a role in ischemic damage, IL-1 elevation has been associated with many neurodegenerative diseases. There is increasing evidence for a role of IL-1 in Alzheimer's disease (AD) (Mrak, R. E. et al. Neurobiol. Aging, Vol. 22, no. 6 (2001), pp. 903-908). Elevated levels of IL-1β, have been shown to surround amyloid plaques in the disease and recent genetic studies have indicated that a polymorphism in IL-1^{α} is linked to an increased risk of AD (3-6 fold increase) (Griffin, W. S. et al. J. Leukoc. Biol. Vol. 72, no. 2 (2002), pp. 233-238). This polymorphism has also been correlated with rate of cognitive decline in AD patients (Murphy, G. M. et al. Neurology, Vol. 56, no. 11 (2001), pp. 1595-1597). The risk of AD is increased even further when the polymorphism in IL-1.alpha. is found in combination with another polymorphism in IL-1β (see Griffin, W. S., supra), providing convincing evidence that these cytokines play an important role in the pathology of the disease.

This assay measures the release of IL-1β from a mouse microglial cell line following an inflammatory challenge with LPS and interferon-gamma. The ability of test articles to inhibit microglial cell activation and IL-1β release is determined by co-incubation of the test article with the inflammatory challenge.

Cerebral ischemic insults are modeled in animals by occluding vessels to, or within, the cranium (Molinari, G. F. in: Barnett, H. J. M. et al. (Eds.), Stroke: Pathophysiology, Diagnosis and Management, Vol. 1 (New York, Churchill Livingstone, 1986). The rat middle cerebral artery occlusion (MCAO) model is one of the most widely used techniques to induce transient focal cerebral ischemia approximating cerebral ischemic damage in humans, e.g., those who suffer from a stroke. The middle cerebral artery used as the ischemic trigger in this model is the most affected vessel in human stroke. The model also entails a period of reperfusion, which typically occurs in human stroke victims. MCAO involving a two-hour occlusion has been found to produce the maximum size of cortical infarction obtainable without increased mortality at twenty-four hours.

### Administration

The compounds of the invention are administered at a therapeutically effective dosage, e.g., a dosage sufficient to provide treatment for the disease states previously described. Administration of the compounds of the invention or the pharmaceutically acceptable salts thereof can be via any of the accepted modes of administration for agents that serve similar utilities.

While human dosage levels have yet to be optimized for the compounds of the invention, a dose may be from about 1 mg to 1 g, preferably 10 mg to 500 mg and most preferably 10 mg to 100 mg per administration. The amount of active compound administered will, of course, be dependent on the subject and disease state being treated, the severity of the affliction, the manner and schedule of administration, and the judgment of the prescribing physician.

In employing the compounds of this invention for treatment of the above conditions, any pharmaceutically acceptable mode of administration can be used. The compounds of this invention can be administered either alone or in combination with other pharmaceutically acceptable excipients, including solid, semi-solid, liquid or aerosol dosage forms, such as, for example, tablets, capsules, powders, liquids, suspensions, suppositories, aerosols or the like. The compounds of this invention can also be administered in sustained or controlled release dosage forms, including depot injections, osmotic pumps, pills, transdermal (including electrotransport) patches, and the like, for the prolonged administration of the compound at a predetermined rate, for example, in unit dosage forms suitable for single administration of precise dosages. The compositions will typically include a conventional pharmaceutical carrier or excipient and a compound of this invention or a pharmaceutically acceptable salt thereof. In addition, these compositions may include other medicinal agents, pharmaceutical agents, carriers, adjuvants, and the like, including, but not limited to, anticoagulants, blood clot dissolvers, permeability enhancers, and slow release formulations.

Generally, depending on the intended mode of administration, the pharmaceutically acceptable composition will contain about 0.1% to 90%, for example about 0.5% to 50%, by weight of a compound or salt of this invention, the remainder being suitable pharmaceutical excipients, carriers, etc.

One manner of administration for the conditions detailed above is oral, using a convenient daily dosage regimen which can be adjusted according to the degree of affliction. For such oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. Such compositions take the form of solutions, suspensions, tablets, dispersible tablets, pills, capsules, powders, sustained release formulations, and the like.

Certain compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose and derivatives thereof, and the like.

Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, sodium acetate, sodium citrate, cyclodextrine derivatives, sorbitan monolaurate, triethanolamine acetate, triethanolamine oleate, etc. Actual methods of preparing such dosage forms are known, or will be apparent, to those skilled in this art; for example, see Remington's Pharmaceutical Sciences, 15^{th} Edition, Easton, Pa., Mack Publishing Company, 1975. The composition or formulation to be administered will, in any event, contain a quantity of the active compound in an amount effective to alleviate the symptoms of the subject being treated. Dosage forms or compositions containing active ingredient in the range of 0.005% to 95% with the balance made up from non-toxic carrier may be prepared.

For a solid dosage form, the solution or suspension in for example, propylene carbonate, vegetable oils or triglycerides, is encapsulated in a gelatin capsule. Such diester solutions, and the preparation and encapsulation thereof, are disclosed in U.S. Pat. Nos. 4,328,245; 4,409,239; and 4,410,545. For a liquid dosage form, the solution, e.g. in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active compound or salt in vegetable oils, glycols, triglycerides, propylene glycol esters (e.g. propylene carbonate) and the like, and encapsulating these solutions or suspensions in hard or soft gelatin capsule shells.

The formulation can be administered in a single unit dosage form for continuous treatment or in a single unit dosage form ad libitum when relief of symptoms is specifically required. For example, the formulation may be administered as a bolus or as a continuous intravenous infusion after onset of symptoms of stroke, myocardial infarction or chronic heart failure.

Another manner of administration is the topical administration. “Topical administration” refers to application of the present compositions by spreading, spraying, etc. onto the surface of the skin. The typical amount applied may vary from about 0.1 mg of composition per square centimeter of skin to about 25 mg of composition per square centimeter of skin. Certain compounds of the present invention may be formulated for topical administration to the epidermis as ointments, creams or lotions, or as transdermal patch. Formulations suitable for topical administration in the mouth include lozenges, pastilles and mouthwashes.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as, for example, sodium acetate, sorbitan monolaurate, triethanolamine oleate, cyclodextrins, etc.

Another approach for parenteral administration employs the implantation of a slow-release or sustained-release system, such that a constant level of dosage is maintained. The percentage of active compound contained in such parenteral compositions is highly dependent on the specific nature thereof, as well as the activity of the compound and the needs of the subject. However, percentages of active ingredient of 0.01% to 10% in solution are employable, and will be higher if the composition is a solid which will be subsequently diluted to the above percentages.

Nasal solutions of the active compound alone or in combination with other pharmaceutically acceptable excipients can also be administered.

Formulations of the active compound or a salt may also be administered to the respiratory tract as an aerosol or solution for a nebulizer, or as a microfine powder for insufflation, alone or in combination with an inert carrier such as lactose. In such a case, the particles of the formulation have diameters of less than 50 microns, for example less than 10 microns.

### EXAMPLES

The following preparations and examples are given to enable those skilled in the art to more clearly understand and to practice the present invention. They should not be considered as limiting the scope of the invention, but merely as being illustrative and representative thereof.

### General Characterization Methods

Nuclear Magnetic Resonance (NMR) spectra is recorded on a Bruker DTX 300 spectrometer using, in most cases, tetramethyl silane (TMS) as the internal reference. Mass spectra is obtained on an Agilent 1100 LC/MSD instrument using either electrospray ionization (positive or negative mode) (ESI) or atmospheric pressure chemical ionization (positive or negative mode) (APCI).

Further, abbreviations used throughout the specification have the following meanings:
DMSO=dimethyl sulfoxide
ELISA=enzyme-linked immunosorbant assay
FBS=fetal bovine serum
h=hour
IC₅₀=The molar concentration of a drug, which produces 50% of the maximum possible inhibition for that drug
M=Molar
M=molar
mg=milligram
min=minute
mL=milliliter
mM=millimolar
mmol=millimole
PBS=phosphate buffered saline
pg=picograms
μL=microliter
μM micromolar

### Example 1

### 5-Lipoxygenase Enzyme Assay

This procedure is used for measuring the enzymatic activity of human recombinant 5-lipoxygenase using a colorimetric method based on the ferric oxidation of xylenol orange.

### Materials

96 well flat bottom microfilter plates (VWR, Catalog # 62402-933 9295)
Lipoxygenase screening assay buffer (Cayman, Catalog # 760710)
Human recombinant 5-Lipoxygenase (Cayman, Catalog # 60402)
Arachidonic Acid (Sigma, Catalog # A3555)
Xylenol orange tetrasodium salt (Aldrich, Catalog # 227854)
Iron (II) sulfate heptahydrate (Sigma, Catalog # F7002)
Sulfuric acid (95-98%) [18 M]
Methanol

### Procedure

Human recombinant 5-lipoxygenase (Cayman Cat # 60402) is used in this assay. The test compound and/or vehicle is added to 0.5/μL 5-lipoxygenase in 50 mM Tris-HCl buffer, pH 7.4. The reaction is initiated by addition of 70 μM arachidonic acid in Tris-HCl buffer, pH 7.4, and terminated after a 10 minute incubation at room temperature by addition of FOX reagent (25 mM sulfuric acid, 100 μM xylenol orange, 100 μM iron (II) sulphate, methanol:water 9:1). The yellow color of acidified xylenol orange is converted to a blue color by the lipid hydroperoxide-mediated oxidation of Fe²⁺ ions and the interaction of the resulting Fe³⁺ ions with the dye. The complex is allowed to form during a 1 hour incubation at room temperature with shaking. Absorbance of the Fe³⁺ complex was then measured at 620 nM using a spectrophotometer.

Negative controls contained enzyme during the incubation step but substrate is not added until after the FOX reagent. Compounds are screened at 5 concentrations in triplicate starting at 10 μM.

Certain compounds of the present invention when tested by this method may show inhibition of 5-Lipoxygenase enzyme.

### Example 2

### 12/15-Lipoxygenase Enzyme Assay

This procedure is used for measuring the enzymatic activity of porcine leukocyte 12/15-lipoxygenase using a colorimetric method based on the ferric oxidation of xylenol orange.

### Materials

96 well flat bottom microfilter plates (VWR, Catalog # 62402-933 9295)
Lipoxygenase screening assay buffer (Cayman, Catalog # 760710)
Porcine leukocyte 12/15-lipoxygenase (Cayman, Catalog # 60300)
Arachidonic Acid (Sigma, Catalog # A3555)
Xylenol orange tetrasodium salt (Aldrich, Catalog # 227854)
Iron (II) sulfate heptahydrate (Sigma, Catalog # F7002)
Sulfuric acid (95-98%) [18 M]
Methanol

### Procedure

Porcine Leukocyte 12/15-lipoxygenase (Cayman Cat # 60300) is used in this assay. Test compound and/or vehicle are added to 1.3 μL 12/15-lipoxygenase in 50 mM Tris-HCl buffer, pH 7.4. The reaction is initiated by addition of 70 μM arachidonic acid in Tris-HCl buffer, pH 7.4, and terminated after a 10 minute incubation at room temperature by addition of FOX reagent (25 mM sulfuric acid, 100 μM xylenol orange, 100 μM iron (II) sulphate, methanol:water 9:1). The yellow color of acidified xylenol orange is converted to a blue color by the lipid hydroperoxide-mediated oxidation of Fe²⁺ ions and the interaction of the resulting Fe³⁺ ions with the dye. The complex is allowed to form during a 1 hour incubation at room temperature with shaking. Absorbance of the Fe³⁺ complex is then measured at 620 nM using a spectrophotometer.

Negative controls contained enzyme during the incubation step but substrate is not added until after the FOX reagent. Compounds are screened at 5 concentrations in triplicate starting at 10 μM.

Certain compounds of the present invention may show inhibition of 12/15-Lipoxygenase enzyme when tested by this method.

### Example 3

### Inhibition of LTB₄ Production in Blood

The following materials are used for this protocol.

### Materials

Human whole blood (Na citrate) (Stanford Blood Center)
A23187, (Sigma, Cat # C-7522)
Leukotriene B₄ EIA reagents (Cayman Chemical, Cat # 520111)
BWA4C (Sigma, Cat # B7559)

### Procedure

### Preparation of A23187:

A23187 is prepared as a 10 mM stock solution in DMSO (aliquots can be stored at −20° C.). On the day of the assay the stock solution is diluted as follows: 70/μL 10 mM stock added to 1.6 mL plasma to give a working concentration of 0.42 mM.

### Preparation of Test Articles:

From a 30 mM stock solution in DMSO, test articles are diluted to a working concentration of 600 μM in PBS (i.e. 10 μl stock solution and 490 μl PBS). This is the highest concentration (gives a final testing concentration of 30 μM). From this 600 μM solution test articles are serially diluted 1:3 in PBS to give a dose-response curve. 1 μl of each concentration of test article is then added to 4 wells of a 96-well plate (i.e. testing in quadruplicate). A positive control compound, BWA4C is used in every assay.

### Blood Stimulation Procedure

Human whole blood is added to the plates containing compounds (190 μl per well) and mixed well. The blood is incubated with compound at 37° C. for 15 minutes. Following this incubation, 10 μl of 0.42 mM A23187 is added to each well except the negative control wells, to give a final calcium ionophore concentration of 20 μM. The plates are then incubated at 37° C. for 60 minutes. After the incubation period, plates are centrifuged for 15 min at 2000 g at 4° C. in sealed microplate buckets. Plasma is then removed for quantitation of LTB₄ levels by ELISA.

### Measurement of LTB₄ Levels by ELISA

LTB₄ levels in the plasma are determined using a commercially available ELISA kit from Cayman Chemicals. The ELISA is run according to the manufacturer's instructions. The LTB₄ levels in the vehicle control sample are then compared to those in which the test article has been added. From this a percent inhibition of LTB₄ production by each concentration of test article is calculated and the IC₅₀ is determined.

Certain compounds of this invention when tested as described above, may show activity in this assay.

### Example 4

### LTB₄-Cell Assay

This procedure is used for measuring the release of the leukotriene LTB₄ from a neutrophil cell line using a competitive ELISA technique.

### Materials and Equipments

Materials for cell preparation and experiment
MPRO cell line (ATCC, Catalog # CRL-11422)
Calcium ionophore (A23187) (Sigma, Catalog # C7522)
Nordihydroguaiaretic acid (NDGA) (BioMol ,Catalog # EI101-0001)
Retinoic Acid (all-trans) (ATRA) (Sigma, Catalog # 95152)
Sterile, tissue-culture treated 96-well plates (Corning, Catalog # 3614)

### Materials for LTB4 ELISA

Precoated (Mouse Anti-Rabbit IgG) EIA 96 Well Strip Plates (Cayman, Catalog # 400004)
Leukotriene B4 AChE Tracer (Cayman Catalog # 420110)
Leukotriene B4 EIA Antiserum (Cayman Catalog # 420112)
Ellman's Reagent (Cayman Catalog # 400050)
EIA Buffer Concentrate (10×) (Cayman Catalog # 400060)
Wash Buffer Concentrate (400×) (Cayman Catalog # 400062)
Plastic plate covers (Cayman Catalog # 400012)

### Procedure

A mouse promyelocytic cell line (MPRO) is used in this assay. These cells are committed immature neutrophils that can be differentiated into mature neutrophils by treatment with 10 μM all-trans retinoic acid for 72 hours.

Following 72 hours of differentiation, cells are stimulated with 1 μM of a calcium ionophore (A23187) in the presence or absence of test compound or vehicle for 1 hour at 37° C. After this time, supernatant is removed from the cells and the LTB₄ levels are determined following manufacturer's instructions, using a Leukotriene B₄ EIA kit from Cayman (Cat # 520111).

The negative controls are media samples from differentiated but unstimulated cells. The compounds are screened at 5 concentrations in quadruplicate starting at 10 μM. Certain compounds of this invention when tested as described above, may show activity in this assay.

### Example 5

Inflammation Assay-Cell-ELAM Assay

Endothelial-Leukocyte Adhesion Molecule (ELAM), also known as E-selectin, is expressed on the surface of endothelial cells. In this assay, lipopolysaccharide (LPS) and IL-1β are used to stimulate the expression of ELAM; test agents are tested for their abilities to reduce this expression, in accordance with studies showing that reduction of leukocyte adhesion to endothelial cell surface is associated with decreased cellular damage (e.g., Takada, M., et al., Transplantation 64: 1520-25, 1997; Steinberg, J. B., et al., J. Heart Lung Trans. 13:306-313, 1994).

Endothelial cells may be selected from any of a number of sources and cultured according to methods known in the art; including, for example, coronary artery endothelial cells, human brain microvascular endothelial cells (HBMEC; Hess, D. C., et al., Neurosci. Lett. 213(1): 37-40, 1996), or lung endothelial cells. Cells are conveniently cultured in 96-well plates. Cells are stimulated by adding a solution to each well containing 10 μg/mL LPS and 100 pg/mL IL-1β for 6 hours in the presence of test agent (specific concentrations and time may be adjusted depending on the cell type). Treatment buffer is removed and replaced with pre-warmed Fixing Solution® (100 μl/well) for 25 minutes at room temperature. Cells are then washed 3×, then incubated with Blocking Buffer (PBS+2% FBS) for 25 minutes at room temperature. Blocking Buffer containing Monoclonal E-Selectin Antibody (1:750, Sigma Catalog #S-9555) is added to each well. Plates are sealed and stored at 4° C. overnight. Plates are washed 4× with 160 μL Blocking Buffer per well. Second Antibody-HRP diluted 1:5000 in Blocking Buffer is then added (100 μL/well), and plates are incubated at room temperature (protected from light) for two hours. Plates are then washed 4X with Blocking Buffer before addition of 100 μL of ABTS Substrate solution at room temperature (Zymed, Catalog #00-2024). Wells are allowed to develop for 35 minutes, before measurement at 402 nm in a Fluoroskan® Reader with shake program for 10 seconds. Positive results are recorded as a decrease in ELAM concentration in tested wells, as compared to control wells.

Certain compounds of this invention when tested as described above, may show activity in this assay.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

1. A compound represented by Formula I: 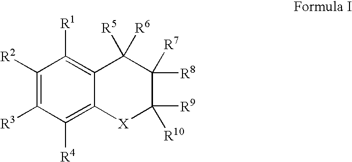
wherein,
X is O, S(O)₀₋₂, or NR;
R¹, R³, and R⁴ are independently selected from the group consisting of hydrogen, C₁-C₁₀ alkyl, C₃-C₆_cycloalkyl, halogen, nitro, cyano, amino, hydroxy, and C₁-C₆ alkoxy,
R² is selected from the group consisting of hydroxy, C₁-C₆ alkoxy, —O-alkenyl, —O-acyl, —O—C(O)-alkylene-COOR^{a}; and —O—C(O)-alkylene-amino;
R⁵ and R⁶ are independently selected from the group consisting of hydrogen, C₁-C₁₀ alkyl, C₃-C₆ cycloalkyl, hydroxy, C₁-C₆ alkoxy, carboxy, C₁-C₁₀ alkylcarbonyl, amino, amido, aminosulfonyl, sulfonylamino, sulfanyl, nitro, cyano, halogen, and —NR^{d}OR^{a}, or together with the carbon atom to which they are attached form C═O, C═NOR^{a}, (C₃-C₈)cycloalkyl ring, an azetidine ring or an oxetan ring;
R⁷ and R⁸ are independently selected from the group consisting of hydrogen, C₁-C₁₀ alkyl, C₃-C₆_cycloalkyl, hydroxy, C₁-C₆ alkoxy, carboxy, C₁-C₁₀ alkylcarbonyl, amino, halogen, and —NR OR^{a}, or together with the carbon atom to which they are attached form C═O, C═NOR^{a}, (C₃-C₈)cycloalkyl ring, an azetidine ring or an oxetan ring;
R⁹ and R¹⁰ are independently selected from the group consisting of hydrogen, C₁-C₁₀ alkyl, C₃-C₆ cycloalkyl, hydroxy, C₁-C₆ alkoxy, carboxy, C₁-C₁₀ alkylcarbonyl, amino, halogen, —NR^{d}OR^{a}, or together with the carbon atom to which they are attached form C═O, C═NOR^{a}, an (C₃-C₈)cycloalkyl ring, an azetidine ring or an oxetan ring;
R is selected from the group consisting of hydrogen, C₁-C₁₀ alkyl, cycloalkyl, and aryl;
R^{a} is selected from the group consisting of hydrogen, C₁-C₁₀ alkyl, cycloalkyl, propylbenzenesulfonamide, and aryl;
R^{d} is hydrogen or C₁-C₁₀ alkyl; and with the proviso that at least one of —CR⁵R⁶, —CR⁷R⁸, and —CR⁹R¹⁰ is an azetidine ring or an oxetan ring; or single stereoisomers, mixtures of stereoisomers, or pharmaceutically acceptable salts thereof.

2. The compound of claim 1, wherein R² is hydroxy, and R¹, R³, and R⁴ are independently hydrogen or C₁-C₁₀ alkyl.

3. The compound of claim 2, wherein —CR⁵R⁶ is an azetidine ring or an oxetan ring.

4. The compound of claim 2, wherein —CR⁷R⁸ is an azetidine ring or an oxetan ring.

5. The compound of claim 2, wherein —CR⁹R¹⁰ is an azetidine ring or an oxetan ring.

6. The compound of claim 2, wherein X is O or S.

7. The compound of claim 6, wherein R⁵ is selected from the group consisting of hydroxy, or —NR^{d}OR^{a},and R⁶ is hydrogen.

8. The compound of claim 2, wherein R⁵ and R⁶ are both hydrogen.

9. The compound of claim 2, wherein X is NR.

10-11. (canceled)

12. A pharmaceutical composition comprising a compound of claim 1, or a pharmaceutically acceptable salt thereof, admixed with a pharmaceutically acceptable excipient.

13. A method of treating a subject with a lipoxygenase mediated condition comprising administering to said subject a therapeutically effective amount of a compound of claim 1.

14. The method of claim 13, wherein the subject is a mammal suffering from diseases selected from the group consisting of apoptosis in cancer cells including prostatic cancer, gastric cancer, breast cancer, pancreatic cancer, colorectal or esophageal cancer and airways carcinoma; diseases involving hypoxia or anoxia including atherosclerosis, myocardial infarction, cardiovascular disease, heart failure (including chronic and congestive heart failure), cerebral ischemia, retinal ischemia, myocardial ischemia, post surgical cognitive dysfunction and other ischemias; diseases involving inflammation, including diabetes, arterial inflammation, inflammatory bowel disease, Crohn's disease, renal disease, pre-menstrual syndrome, asthma, allergic rhinitis, gout, cardiopulmonary inflammation, rheumatoid arthritis, osteoarthritis, muscle fatigue and inflammatory disorders of the skin including acne, dermatitis and psoriasis; disorders of the airways including asthma, chronic bronchitis, human airway carcinomas, mucus hypersecretion, chronic obstructive pulmonary disease (COPD) pulmonary fibrosis caused by chemotherapy or other drugs, idiopathic pulmonary fibrosis, cystic fibrosis and adult respiratory distress syndrome; diseases involving central nervous system (CNS) disorders including psychiatric disorders including anxiety and depression; neurodegeneration and neuroinflammation including Alzheimer's, dementia and Parkinson's disease; peripheral neuropathy including spinal chord injury, head injury and surgical trauma, and allograft tissue and organ transplant rejection; diseases involving the autoimmune system including psoriasis, eczema, rheumatoid arthritis, diabetes; and disorders involving bone loss or bone formation.

15. The method of claim 13, wherein the subject is a mammal suffering from diabetes, arthritis, rheumatoid arthritis, chronic obstructive pulmonary disease (COPD), asthma, allergic rhinitis, or atherosclerosis.
